(19) United States
(12) Patent Application Publication
(10) Pub. No.: US 2025/0235275 A1
(43) Pub. Date: Jul. 24, 2025

(54) MECHANICAL WORKAROUND TWO-WAY FOOTSWITCH FOR A SURGICAL ROBOTIC SYSTEM

(71) Applicant: Covidien LP, Mansfield, MA (US)

(72) Inventors: Graham J. Barrett, Denver, CO (US); Sara A. Malang, Longmont, CO (US); Robert J. Stephens, Superior, CO (US); Chelsea E. Walbridge, Longmont, CO (US); Christopher T. Tschudy, Arvada, CO (US); Dylan R. Kingsley, Broomfield, CO (US); Daniel A. Joseph, Golden, CO (US); James D. Allen, IV, Broomfield, CO (US)

(21) Appl. No.: 18/839,787

(22) PCT Filed: Mar. 21, 2023
(86) PCT No.: PCT/IB2023/052763

§ 371 Date: Aug. 20, 2024

Related U.S. Application Data
(60) Provisional application No. 63/323,115, filed on Mar. 24, 2022.

(51) Int. Cl. A61B 34/35; A61B 17/00; A61B 34/30

(52) U.S. Cl. CPC A61B 34/35; A61B 17/0057; A61B 2017/00973; A61B 2034/305

(57) ABSTRACT

Devices to control a movement or instrument function of a robotic arm of a surgical robotic system include a first foot pedal with a button, a second foot pedal with a button, and a connector. The connector includes a first and second end and a top and bottom side. The top side of the first end is attached to a bottom side of the button of the first foot pedal, and the second end is configured to be positioned under a bottom side of the button of a second foot pedal to interlock the first foot pedal with the second foot pedal. When a force moves the button of the second foot pedal to a position, the button of the second foot pedal moves the connector which subsequently moves the button of the first foot pedal to an activation position to send an input signal to a surgical console.

### BACKGROUND

Surgical robotic systems may include a surgical console controlling one or more surgical robotic arms, each having a surgical instrument having an end effector (e.g., forceps or grasping instrument). In operation, a user provides input to the surgical robotic systems through one or more interface devices, which are interpreted by a control tower of a surgical console as movement commands for moving the surgical robotic arm. Based on the user inputs, the surgical console sends movement commands to the robotic arm so that the robotic arm is moved to a position over a patient and the surgical instrument is guided into a small incision via a surgical access port or a natural orifice of a patient to position the end effector at a work site within the patient's body.

### SUMMARY

This disclosure describes devices, systems, and methods to control a movement or instrument function of a robotic arm of a surgical robotic system. One embodiment of the present disclosure is a device for a surgical robotic system. The device includes a first foot pedal with a button including a bottom side. The device includes a second foot pedal with a button including a bottom side. The device includes a connector. The connector includes a first end, a second end, a top side, and a bottom side. A top side of the first end is attached to the bottom side of the button of the first foot pedal, and the second end is configured to be positioned under the bottom side of the button of a second foot pedal to interlock the first foot pedal with the second foot pedal. When a force moves the button of the second foot pedal to a position, the button of the second foot pedal moves the connector, and the connector moves the button of the first foot pedal to an activation position.

In aspects, the first foot pedal is configured to generate an input signal in response to the first foot pedal being moved to the activation position, the input signal corresponding to at least one of a movement command of an arm of the surgical robotic system or an instrument function.

In aspects, the first foot pedal is further configured to send the input signal to a surgical console configured to remotely control an arm or an instrument function of the surgical robotic system based on the first input signal.

In aspects, the instrument function includes at least one of vessel sealing, bipolar coagulation, tissue cutting, stapling, monopolar power level, or ultrasonic power level.

In aspects, the first foot pedal is configured to generate a physical click in response to the first foot pedal being moved to the activation position.

In aspects, the first foot pedal includes a light, and the light is configured to illuminate in response to the first foot pedal being moved to the activation position.

In aspects, the first foot pedal includes a speaker configured to generate a noise in response to the first foot pedal being moved to the activation position.

In aspects, the connector includes a hook, a clip, a clasp, or a snap, to interlock the connector with a support column of the second foot pedal.

In aspects, the connector is configured to rotate with respect to an axis of the bottom side of the button of the first foot pedal to unlock the first foot pedal from the second foot pedal.

Another embodiment of the present disclosure is system to mechanically interlock a first foot pedal with a second foot pedal. The system includes a first foot pedal including a first button, a first support column, and a first base, and a second foot pedal including a second button, a second column, a second base, and a connector. The connector includes a first end, a second end, a top side, and a bottom side. The first end of the connector is attached to the second button of the second foot pedal and the second end of the connector is configured to be positioned under a bottom side of the first button of the first foot pedal to interlock the second foot pedal with the first foot pedal. When a force moves the first button of the first foot pedal to a position, the first button of the first foot pedal moves the connector, and the connector moves the second button of the second foot pedal to an activation position.

Another embodiment of the present disclosure is a device to mechanically connect a first foot pedal of a surgical robotic system with a second foot pedal of the surgical robotic system. The device includes a connector with a first end, a second end, a top side, and a bottom side. A top side of the first end of the connector is attached to a bottom side of a button of the first foot pedal and the second end of the connector is positioned under a bottom side of a button of the second foot pedal to interlock the first foot pedal with the second foot pedal. When a force moves the button of the second foot pedal to a position, the button of the second foot pedal moves the connector, and the connector moves the button of the first foot pedal to an activation position.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5a is a side view of a first foot pedal of the surgical robotic system of FIG. 1 in an open position and a second foot pedal of the surgical robotic system of FIG. 1 in an open position according to an embodiment of the present disclosure;

FIG. 5b is a side view of a first foot pedal of the surgical robotic system of FIG. 1 in an activation position and a second foot pedal of the surgical robotic system of FIG. 1 in an open position according to an embodiment of the present disclosure;

FIG. 5c is a side view of a first foot pedal of the surgical robotic system of FIG. 1 in a second position and second foot pedal of the surgical robotic system of FIG. 1 in an activation position according to an embodiment of the present disclosure; and

FIG. 6 is a side view of a first foot pedal of the surgical robotic system of FIG. 1 in a second position and second foot pedal of the surgical robotic system of FIG. 1 in an open position according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgical console, a control tower, and one or more mobile carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

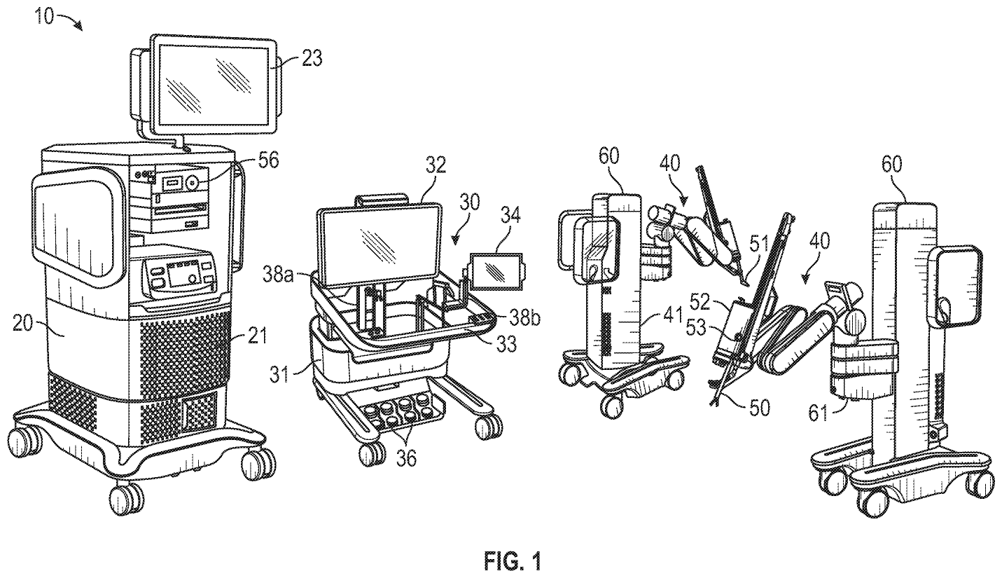

With reference to FIG. 1, a surgical robotic system 10 includes a control tower 20, which is connected to all of the components of the surgical robotic system 10 including a surgeon console 30 and one or more mobile carts 60. Each of the mobile carts 60 includes a robotic arm 40 having a surgical instrument 50 removably coupled thereto. The robotic arms 40 is also coupled to the mobile cart 60. The system 10 may include any number of mobile carts 60 and/or robotic arms 40.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device configured to receive video feed from the endoscopic camera 51, perform image processing, and output the processed video stream.

The surgeon console 30 includes a first display 32, which displays a video feed of the surgical site provided by camera 51 of the surgical instrument 50 disposed on the robotic arm 40, and a second display 34, which displays a user interface for controlling the surgical robotic system 10. The first and second displays 32 and 34 are touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instruments 50 execute a desired movement sequence in response to input from the foot pedals 36 and the handle controllers 38a and 38b.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

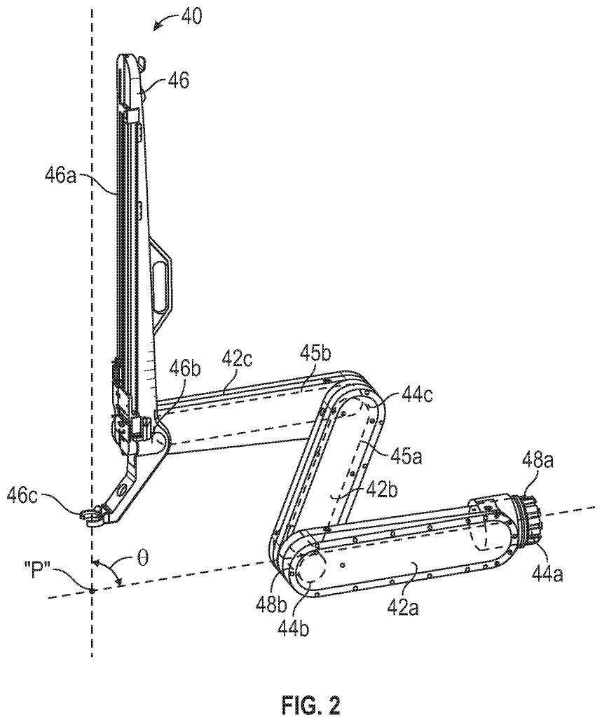

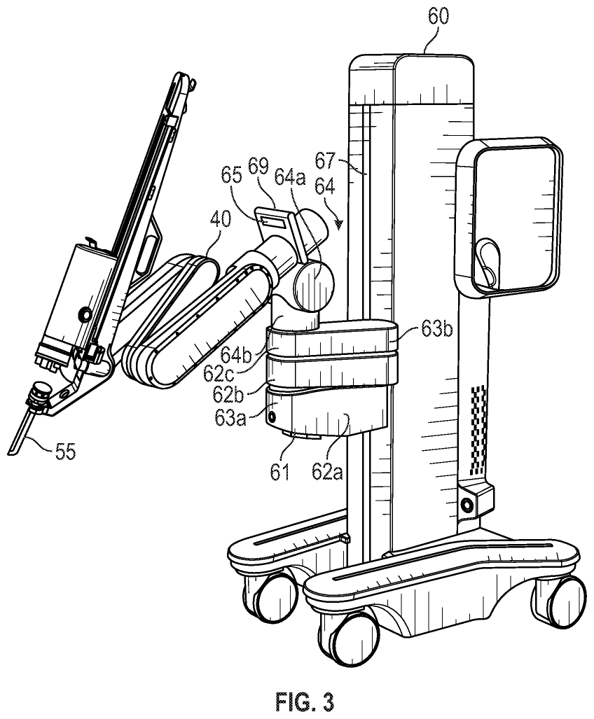

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and is configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic port 55 (FIG. 3) held by the holder 46. The holder 46 also includes a port latch 46c for securing the port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

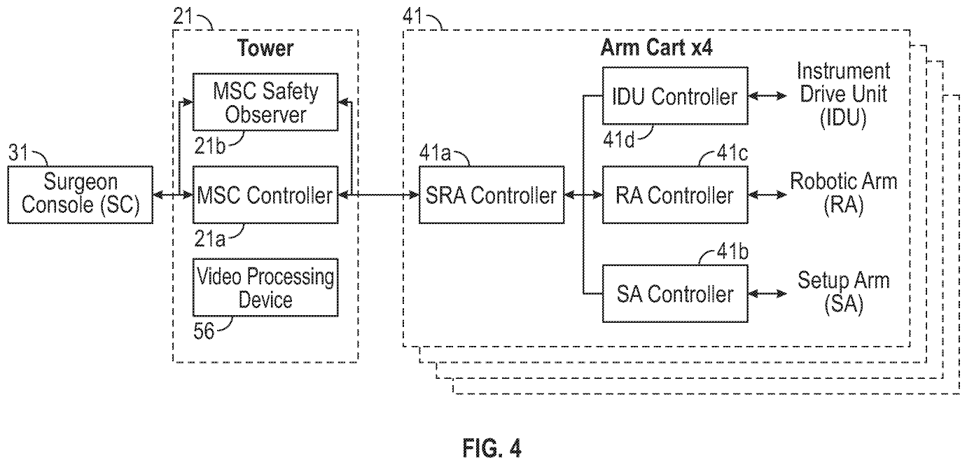

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the instrument drive unit 52 and communicates these to the computer 41 of the robotic arm 40. The controller 21a also receives back the actual joint angles and uses this information to determine force feedback commands that are transmitted back to the computer 31 of the surgical console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the instrument drive unit 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the instrument drive unit 52. The IDU controller 41d calculates actual angles based on the motor positions and transmits the actual angles back to the main cart controller 41a.

The robotic arm 40 is controlled as follows. Initially, a pose of the handle controller controlling the robotic arm 40, e.g., the handle controller 38a, is transformed into a desired pose of the robotic arm 40 through a hand eye transform function executed by the controller 21a. The hand eye function, as well as other functions described herein, is/are embodied in software executable by the controller 21a or any other suitable controller described herein. The pose of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In some instances, the coordinate position may be scaled down and the orientation may be scaled up by the scaling function. In addition, the controller 21a also executes a clutching function, which disengages the handle controller 38a from the robotic arm 40. In particular, the main cart controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, and 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

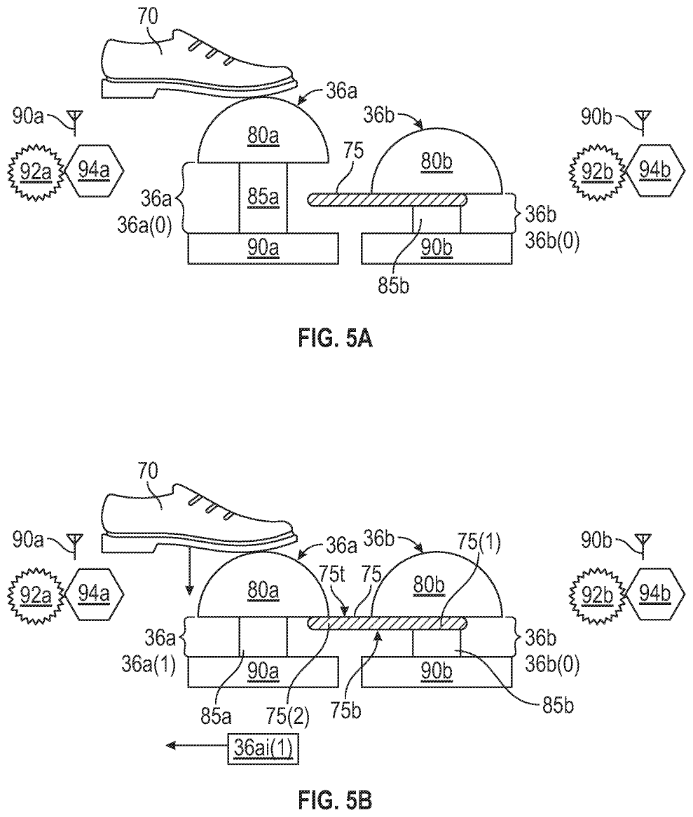

With reference to FIGS. 5a-5c, surgical robotic system 10 of FIG. 1 may include a first foot pedal 36a and a second foot pedal 36b. Foot pedal 36a may be configured with an open position 36a(0), an activation position 36a(1), and a second position 36a(2) and may control a function of an instrument during a surgery, for example, controlling vessel sealing during ligation of blood vessels. Foot pedal 36b may be configured with an open position 36b(0) and an activation position 36b(1) and may control a function of an instrument during a surgery, for example, controlling cutting during ligation of blood vessels. As discussed in more detail below, foot pedals 36a and 36b may function as a mechanical two-way footswitch and may be aligned and/or mechanically connected so that a foot 70 of a user may be able to move foot pedal 36b when applying a force to foot pedal 36a.

FIGS. 5a-5c are side views of various positions of foot pedals 36a and 36b. FIG. 5a is a side view of foot pedal 36a in an open position and foot pedal 36b in an open position. Foot switch 36a may include a button 80a, a support column 85a, and a base 90a. Foot switch 36b may include a button 80b, a support column 85b, a base 90b, and a connector 75. Buttons 80a and 80b may be attached to and/or supported by support columns 85a and 85b respectively, and support columns 85a and 85b may be attached to bases 90a and 90b, respectively. Button 80a of foot pedal 36a may be configured to be supported by support column 85a in an open position 36a(0) when no force is applied to button 80a by a foot 70 of a user. Foot switch 36a may not generate an input signal when button 80 is not moved and foot switch 36a is in open position 36a(0).

Button 80b of foot pedal 36b may be configured to be supported by support column 85b in an open position 36b(0) when no force is applied to button 80b or connector 75. Connector 75 may include a top side 75t, a bottom side 75b a first end 75 (1) and a second end 75 (2). Top side 75t of first end 75 (1) may be attached to a bottom side of button 80b and may move button 80b when connector 75 is moved. Foot pedal 36b may be positioned adjacent to foot pedal 36a so that foot pedal 36b is aligned with foot pedal 36a and second end 75 (2) of connector 75 is positioned underneath button 80a of foot pedal 36a. As described in more detail below, when second end 75 (2) of connector 75 is positioned under button 80a, connector 75 may mechanically interlock foot pedal 36b with foot pedal 36a and connector 75 may be moved by button 80a when button 80a is moved. Foot switch 36b may not generate an input signal when button 80b or connector 75 is not moved and foot switch 36b is in open position 36b(0).

FIG. 5b is a side view of foot pedal 36a in an activation position and foot pedal 36b in open position 36b(0). Button 80a of foot pedal 36a may be configured to move to an activation position 36a(1) when a force is applied to move button 80a to activation position 36a(1) by a foot 70 of a user. Activation position 36a(1) may be above a height of second end 75 (2) of connector 75 and when button 80a of foot pedal 36a is moved to activation position 36a(1), a bottom side of button 80a may not contact second end 75 (2) of connector 75. Button 80b of foot pedal 36b may remain in open position 36b(0) when button 80a of foot pedal 36a is moved to activation position 36a(1) and no force is applied to connector 75. Foot switch 36b may not generate an input signal when foot switch 36b is in open position 36b(0).

Foot switch 36a may be configured to generate tactile feedback to a user such as a physical click or vibration when button 80a is moved to activation position 36a(1). Foot switch 36a may also generate a noise by a speaker 92a and/or illuminate a light 94a when button 80a is moved to activation position 36a(1). Activation position 36a(1) may be associated with a specific movement or instrument function of surgical robotic system 10. Foot switch 36a may generate a first input signal 36ai(1) when button 80a is moved to activation position 36a(1). Foot switch 36a may send first input signal 36ai(1) to surgical console 30, control tower 20 and/or at least one of the robotic arms 40 of FIG. 1 by a transmitter 95a of foot switch 36a. While a wireless transmitter 95a is illustrated, it is contemplated and within the scope of the disclosure that hardwired connections may be used to send first input signal 36ai(1) to the surgical console 30, the control tower 20 and/or the robotic arm(s) 40.

First input signal 36ai(1), from foot switch 36a, may be utilized by surgical console 30 to remotely control a specific movement or instrument function of surgical robotic system 10. Instrument function, remotely controlled by first input signal 36ai(1) from foot switch 36a, may include vessel sealing, bipolar coagulation, tissue cutting, stapling, monopolar power level, ultrasonic power level, etc. Instrument function, remotely controlled by first input signal 36ai(1) from foot switch 36a, may be for a predetermined period of time, for example, first input signal 36ai(1) may initiate bipolar coagulation, which may be activated for a predetermined amount of time.

FIG. 5c is a side view of foot pedal 36a in a second position and foot pedal 36b in an activation position. Button 80a of foot pedal 36a may be configured to move to a second position 36a(2) when a force is applied to move button 80a to second position 36a(2) by a foot 70 of a user. A user may move button 80a of foot pedal 36a to second position 36a(2) when instrument function controlled by first input signal 36ai(1) is complete. Foot switch 36a may further be configured to generate tactile feedback to the user when instrument function controlled by first input signal 36ai(1) is complete. Tactile feedback may include a physical click or vibration, a noise or stop of a noise by speaker 92a, illumination or stop of illumination of light 94a. When button 80a of foot pedal 36a is moved to second position 36a(2), a bottom side of button 80a may contact and move connector 75. Connector 75, interlocked with foot pedal 36a and attached to button 80b of foot pedal 36b, may move button 80b of foot pedal 36b to activation position 36b(1) when connector 75 is moved by button 80a of foot pedal 36a moving to second position 36a(2). Connector 75 may be configured as a straight rod with first end 75 (1) attached to button 80b of foot pedal 36b and second end 75 (2) positioned under button 80a of foot pedal 36a to interlock foot pedal 36b with foot pedal 36a. In another embodiment, connector 75 may include a coupling device at second end 75 (2) which may secure connector 75 with support column 85a to interlock foot pedal 36b with foot pedal 36a. For example, connector 75 may include a hook, a clip, a clasp, or a snap to interlock connector 75 with support column 85a. In another example, connector 75 may interlock with a grove or channel along a surface of column 85a.

When button 80b of foot pedal 36b is moved to activation position 36b(1), foot pedal 36b may generate tactile feedback to a user. Tactile feedback may include a physical click or vibration, a noise by speaker 92b, and/or illuminate light 94b. A noise and/or illumination generated by foot pedal 36b may be different for activation position 36b(1) than for activation position 36a(1) of foot pedal 36a so that a user can easily distinguish between the two foot pedals. For example, foot pedal 36b may illuminate light 94b when button 80b is moved to activation position 36b(1) and foot pedal 36a may generate a buzzer noise by speaker 92a when button 80a is moved to activation position 36a(1). Activation position 36b(1) may be associated with a specific movement or instrument function of robotic arms 40 of FIG. 1. Foot pedal 36b may generate a first input signal 36bi(1) when button 80b is moved to activation position 36b(1) and foot pedal 36b may send first input signal 36bi(1) to surgical console 30, control tower 20 and/or at least one of the robotic arms 40 of FIG. 1 by transmitter 95b of foot pedal 36b. As previously stated, hardwired connections may be used to send first input signal 36bi(1) to the surgical console 30 the control tower 20 and/or the robotic arm(s) 40. First input signal 36bi(1), from foot pedal 36b, may be utilized by surgical console 30 to remotely control a specific movement or instrument function of robotic arms 40 of FIG. 1. Instrument function remotely controlled by first input signal 36bi(1) from foot pedal 36b may include vessel sealing, bipolar coagulation, tissue cutting, stapling, monopolar power level, ultrasonic power level, etc.

As shown in FIGS. 5a-5c, foot pedal 36a and foot pedal 36b may function as a mechanical two-way footswitch for surgical robotic system 10. Connector 75 may be attached to button 80b of foot pedal 36b and positioned underneath button 80a of foot pedal 36a. Foot 70 of a user may apply a force to button 80a of foot pedal 36a to move foot pedal 36a to an activation position and generate first input signal 36ai(1) to remotely control a first specific movement or instrument function of robotic arms 40 of FIG. 1. Foot 70 of a user may apply a force to button 80a of foot pedal 36a to move foot pedal 36a to a second position and button 80a may move connector 75 and button 80b of foot pedal 36b to activation position 36b(1) and generate first input signal 36bi(1) to remotely control a second specific movement or instrument function of robotic arms 40 of FIG. 1.

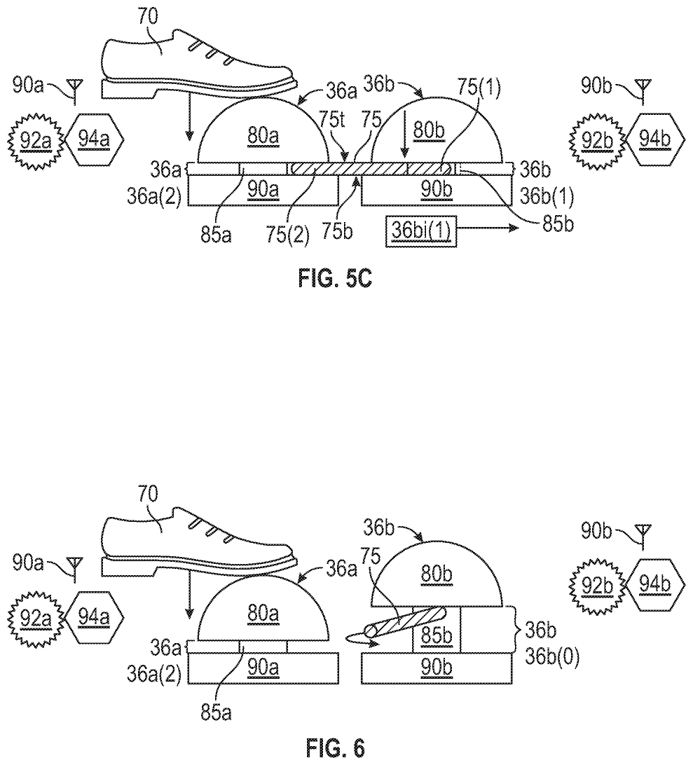

FIG. 6 is a side view of foot pedal 36a in second position 36a(2) and foot pedal 36b in open position 36b(0). As previously shown in FIG. 5c, connector 75 of foot pedal 36b may be positioned underneath button 80a to interlock foot pedal 36b with foot pedal 36a. Connector 75 may be configured to swing or rotate with respect to an axis of a bottom side of button 80b or support column 85b to unlock or decouple foot pedal 36b from foot pedal 36a. When connector 75 is not positioned underneath button 80a, and foot pedal 36b is unlocked or decoupled, from foot pedal 36a, connector 75 may not move button 80b of foot pedal 36b to activation position 36b(1) when button 80a of foot pedal 36a is moved to second position 36a(2).

A device in accordance with the present disclosure may provide a user with the ability to control more than one movement or instrument function of robotic arms 40 of a robotic surgical system 10 with a first foot pedal mechanically interlocked with a second foot pedal. A device in accordance with the present disclosure may provide a user with the ability to control different movements or instrument functions of robotic arm 40 of robotic surgical system 10 by depressing a first foot pedal to either a first activation position or a second position to activate a second foot pedal mechanically interlocked with the first foot pedal. A device in accordance with the present disclosure may provide a user with the ability to control different movements or instrument functions of the robotic arm of a robotic surgical system by depressing a first foot pedal to either and activation position or a second position to activate a second foot pedal. A device in accordance with the present disclosure may provide a user with indicator when a first or second foot pedal is moved to an activation position.

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

What is claimed is:

1. A device for a surgical robotic system, the device comprising:
a first foot pedal with a button including a bottom side;
a second foot pedal with a button including a bottom side;
a connector including a first end, a second end, a top side, and a bottom side, a top side of the first end of the connector being attached to the bottom side of the button of the first foot pedal and the second end of the connector is configured to be positioned under the bottom side of the button of the second foot pedal to interlock the first foot pedal with the second foot pedal, wherein when a force moves the button of the second foot pedal to a position, the button of the second foot pedal moves the connector, and the connector moves the button of the first foot pedal to an activation position.

2. The device of claim 1, wherein the first foot pedal is configured to generate an input signal in response to the first foot pedal being moved to the activation position, the input signal corresponding to at least one of a movement command of an arm of the surgical robotic system or an instrument function.

3. The device of claim 2, wherein the first foot pedal is further configured to send the input signal to a surgical console configured to remotely control an arm or an instrument function of the surgical robotic system based on the first input signal.

4. The device of claim 3, wherein the instrument function includes at least one of vessel sealing, bipolar coagulation, tissue cutting, stapling, monopolar power level, or ultrasonic power level.

5. The device of claim 1, wherein the first foot pedal is configured to generate a physical click in response to the first foot pedal being moved to the activation position.

6. The device of claim 1, further comprising a light configured to illuminate in response to the first foot pedal being moved to the activation position.

7. The device of claim 1, further comprising a speaker configured to generate a noise in response to the first foot pedal being moved to the activation position.

8. The device of claim 1, wherein the connector includes a hook, a clip, a clasp, or a snap to interlock the connector with a support column of the second foot pedal.

9. The device of claim 1, wherein the connector is configured to rotate with respect to an axis of the bottom side of the button of the first foot pedal to unlock the first foot pedal from the second foot pedal.

10. A system to mechanically interlock a first foot pedal with a second foot pedal, the system comprising:
a first foot pedal including a first button, a first support column, and a first base; and
a second foot pedal including a second button, a second support column, a second base, and a connector,
wherein the connector includes a first end, a second end, a top side, and a bottom side, the first end of the connector is attached to the second button of the second foot pedal and the second end of the connector is configured to be positioned under a bottom side of the first button of the first foot pedal to interlock the second foot pedal with the first foot pedal,
wherein when a force moves the first button of the first foot pedal to a position, the first button of the first foot pedal moves the connector, and the connector moves the second button of the second foot pedal to an activation position.

11. The system of claim 10, wherein the second foot pedal is configured to generate an input signal in response to the second foot pedal being moved to the activation position, the input signal corresponding to at least one of a movement command of an arm of a surgical robotic system or an instrument function.

12. The system of claim 11, wherein the second foot pedal is further configured to send the input signal to a surgical console configured to remotely control an arm or an instrument function of the surgical robotic system based on the first input signal.

13. The system of claim 12, wherein the instrument function includes at least one of vessel sealing, bipolar coagulation, tissue cutting, stapling, monopolar power level, or ultrasonic power level.

14. The system of claim 10, wherein the second foot pedal is configured to generate a physical click in response to the second foot pedal being moved to the activation position.

15. The system of claim 10, wherein the second foot pedal further comprises a light configured to illuminate in response to the second foot pedal being moved to the activation position.

16. The system of claim 10, wherein the connector includes a hook, a clip, a clasp, or a snap to interlock the connector with the support column of the second foot pedal.

17. The system of claim 10, wherein the connector is configured to rotate with respect to an axis of a bottom side of the button of the first foot pedal to unlock the first foot pedal from the second foot pedal.

18. A device to mechanically connect a first foot pedal of a surgical robotic system with a second foot pedal of the surgical robotic system, the device comprising:
a connector with a first end, a second end, a top side, and a bottom side, wherein a top side of the first end of the connector is attached to a bottom side of a button of the first foot pedal and the second end is positioned under a bottom side of a button of the second foot pedal to interlock the first foot pedal with the second foot pedal,
wherein when a force moves the button of the second foot pedal to a position, the button of the second foot pedal moves the connector, and the connector moves the button of the first foot pedal to an activation position.

19. The device of claim 18, wherein the connector includes a hook, a clip, a clasp, or a snap to interlock the connector with a support column of the second foot pedal.

20. The device of claim 1, wherein the connector is configured to rotate with respect to an axis of the bottom side of the button of the first foot pedal to unlock the first foot pedal from the second foot pedal.
